(19) United States
(12) Patent Application Publication
(10) Pub. No.: US 2025/0356626 A1
(43) Pub. Date: Nov. 20, 2025

(54) METHOD FOR CREATING PREDICTION MODEL FOR CORROSION OF METALS AND METHOD FOR PREDICTING METAL CORROSION

(71) Applicant: KABUSHIKI KAISHA KOBE SEIKO SHO (KOBE STEEL, LTD.), Kobe-shi (JP)

(72) Inventors: Ryouhei KINOSHITA, Kobe-shi (JP); Yuya TAKARA, Kobe-shi (JP); Yasumasa NAKADOI, Kobe-shi (JP); Takahiro OZAWA, Kobe-shi (JP)

(73) Assignee: KABUSHIKI KAISHA KOBE SEIKO SHO (KOBE STEEL, LTD.), Kobe-shi (JP)

(21) Appl. No.: 19/191,564

(22) Filed: Apr. 28, 2025

(30) Foreign Application Priority Data
May 20, 2024 (JP) 2024-081960

(51) Int. Cl. G06V 10/764; G01N 21/88; G01N 33/20; G06T 7/00; G06T 7/40; G06T 7/90; G06V 20/70

(52) U.S. Cl. CPC G06V 10/764; G01N 21/8851; G01N 33/20; G06T 7/0002; G06T 7/40; G06T 7/90; G06V 20/70; G06T 2207/10024; G06T 2207/20016; G06T 2207/20081; G06T 2207/30136; G06V 2201/06

(57) ABSTRACT

A method for creating a prediction model for corrosion of metals includes using as training data a plurality of label images which indicate a distribution of substances present in a corrosion process of a metal surface, in terms of a distribution of labels, in a plurality of time-series visible light images indicating the corrosion process on the metal surface to create a prediction model for corrosion of metals which predicts a future change in a corrosion state of a metal surface to be predicted from a visible light image of the metal surface. The number of the labels is set according to the number of types of the substances. The distribution of the labels is created based on a color distribution of coordinate values in color space of the visible light images.

### BACKGROUND OF THE INVENTION

### 1. Field of the Invention

The present invention relates to a method for creating a prediction model for corrosion of metals and to a method for predicting metal corrosion.

### 2. Description of the Related Art

Known methods for predicting the corrosion state of a steel by machine learning include, for example, the technique described in Japanese Unexamined Patent Application Publication No. 2018-136124. The technique of Japanese Unexamined Patent Application Publication No. 2018-136124 involves extracting, from weathering steels stored in a weathering steel database, a plurality of weathering steels having corrosion images similar to a corrosion image of a weathering steel to be guaranteed and, based on the appearance ratings of the plurality of weathering steels, determining the appearance rating of the weathering steel to be guaranteed. The technique also involves determining a corrosion environment (a time-average amount of airborne salt, and preferably also the number of dry-wet cycles) for the weathering steel to be guaranteed based on a corrosion environment (a time-average amount of airborne salt, and preferably also the number of dry-wet cycles) for the plurality of weathering steels.

### SUMMARY OF THE INVENTION

The technique described in Japanese Unexamined Patent Application Publication No. 2018-136124 requires input of not only an image of a steel to be predicted, but also associated conditions such as the material of the steel and environmental exposure time. The prediction target is a time-dependent change in the appearance rating of the steel in an exposure environment; it is difficult to predict a time-dependent change in the entire steel surface. Thus, there is room for improvement in terms of simple and highly accurate prediction of a time-dependent change in the corrosion state of a metal surface.

It is an object of the present invention to predict a time-dependent change in the corrosion state of a metal surface simply with high accuracy.

The present invention provides a method for creating a prediction model for corrosion of metals, comprising using as training data a plurality of label images which indicate a distribution of substances, in terms of a distribution of labels, in a plurality of time-series visible light images indicating a corrosion process on a metal surface to create a prediction model for corrosion of metals which predicts a future change in a corrosion state of a metal surface to be predicted from a visible light image of the metal surface, wherein the number of the labels is set according to the number of types of the substances present in the corrosion process of the metal surface, and wherein the distribution of the labels is created based on a color distribution of coordinate values in color space of the visible light images.

This method uses, as training data, a label image that indicates a distribution of labels in place of a distribution of substances, which is seen as a color distribution in a visible light image of a metal surface. In particular, the pixel value of each pixel in the visible light image of the metal surface is replaced with a label set according to a substance produced on the metal surface during a corrosion process. Thus, the method predicts not the corrosion state of an unspecified number of substances, but the corrosion state of a small number of specified substances. This enables a highly accurate prediction. Further, a prediction model for corrosion of metals can be created by preparing information on the types of substances which are to be precipitated during a corrosion process on the metal surface and which have been identified by any method in advance, and time-series visible light images which indicate the corrosion process on the metal surface. Thus, the method can be performed in a very simple manner. In particular, the method directly learns and predicts a time-dependent change in the label distribution, and therefore does not require information on the material of the metal surface, the corrosion environment, etc.

The color distribution may be created by classifying multi-dimensional coordinate values in color space of the visible light images into a predetermined number of classes for each dimension by machine learning.

This method uses a color distribution obtained by classifying coordinate values in color space, which can be a huge amount of data, into a predetermined number of classes. This can facilitate handling of the data. For example, when three-dimensional RGB (red, green, blue) coordinate values in color space constitute each pixel of a visible light image with 256 levels (0 to 255) for each dimension, a color distribution may be used which uses three discrete classes of low intensity (0-84), medium intensity (85-169), and high intensity (170-255) for each of RGB (red, green, blue) dimensions. The color distribution has three classes for R (red), three classes for G (green), and three classes for B (blue), thus having 27 classes or variations in total.

The machine learning may be unsupervised learning.

The use of unsupervised learning enables classification which is less susceptible to variations in brightness and color tone among images and which is highly tolerant of changes in the imaging environment. In other words, since visible light images may vary in brightness and color tone depending on the imaging environment, the use of supervised learning may result in incorrect classification due to a change in the imaging environment.

The present invention also provides a method for predicting metal corrosion, comprising a preparation step of preparing a learned prediction model for corrosion of metals prediction model for corrosion of metals using as training data a plurality of label images which indicate a distribution of substances, in terms of a distribution of labels, in a plurality of time-series visible light images indicating a corrosion process on a metal surface, wherein the number of the labels is set according to the number of types of the substances present in the corrosion process of the metal surface, and wherein the distribution of the labels is created based on a color distribution of coordinate values in color space of the visible light images;
a color distribution acquisition step of acquiring a color distribution based on coordinate values in color space from a visible light image indicating a corrosion process on a metal surface to be predicted;
a labeling step of acquiring a label image which indicates a distribution of labels created based on the color distribution; and
a corrosion prediction step of inputting the label image to the prediction model for corrosion of metals to predict a future change in a corrosion state of the metal surface.

According to this method, as with the above-described method, a time-dependent change in the corrosion state of a metal surface can be predicted simply with high accuracy.

The color distribution may be created by classifying multi-dimensional coordinate values in color space of the visible light image into a predetermined number of classes for each dimension by machine learning.

As with the above-described method, this method uses a color distribution obtained by classifying coordinate values in color space into a predetermined number of classes. This can facilitate handling of the data.

The machine learning may be unsupervised learning.

As with the above-described method, the use of unsupervised learning enables classification which is less susceptible to variations in brightness and color tone among images and which is highly tolerant of changes in the imaging environment.

The method for predicting metal corrosion may further comprise a coarse-pixelation step of dividing the label image acquired in the labeling step into a plurality of coarse pixel regions, and taking a label with a highest frequency of appearance among labels contained in each of the coarse pixel regions as a label of that coarse pixel region.

This method can reduce the complexity of the label distribution, making it possible to perform simpler estimation.

According to the present invention, a time-dependent change in the corrosion state of a metal surface can be predicted simply with high accuracy.

### BRIEF DESCRIPTION OF THE DRAWINGS

FIG. 1 is a flowchart showing a method for creating a prediction model for corrosion of metals according to an embodiment of the present invention;

FIG. 2 shows visible light images of a steel surface 1, 3, 6, 8, 24, 48, and 72 hours after the start of corrosion;

FIG. 3 shows label images of the steel surface 1, 3, 6, 8, 24, 48, and 72 hours after the start of corrosion;

FIG. 4 is a diagram illustrating coarse-pixelation of a label image;

FIG. 5 shows coarsely pixelated label images of the steel surface 1, 3, 6, 8, 24, 48, and 72 hours after the start of corrosion;

FIG. 6 is a flowchart showing a metal corrosion prediction method using a prediction model for corrosion of metals;

FIG. 7 shows images illustrating examples of the results of prediction by a prediction model for corrosion of metals;

FIG. 8 is a graph showing the occupied area ratio of each label in an experimental steel; and

FIG. 9 is a graph showing the occupied area ratio of each label in predicted results.

### DESCRIPTION OF THE PREFERRED EMBODIMENTS

Embodiments of the present invention will now be described with reference to the attached drawings.

In the present embodiments, a method for creating a prediction model for corrosion of metals, which predicts a future change in the corrosion state of a metal surface from visible light images of the metal surface, will be described first, followed by a description of a metal corrosion prediction method using the prediction model for corrosion of metals.

The following description illustrates by way of example a method for creating a prediction model for corrosion of metals and a method for predicting metal corrosion, both in a case in which corrosion of a steel surface occurs due to salt spray. The type of a metal to be predicted, the corrosion environment, the corrosion period, and the measurement interval are not particularly limited; any metal, any corrosion environment, and any corrosion period may be used.

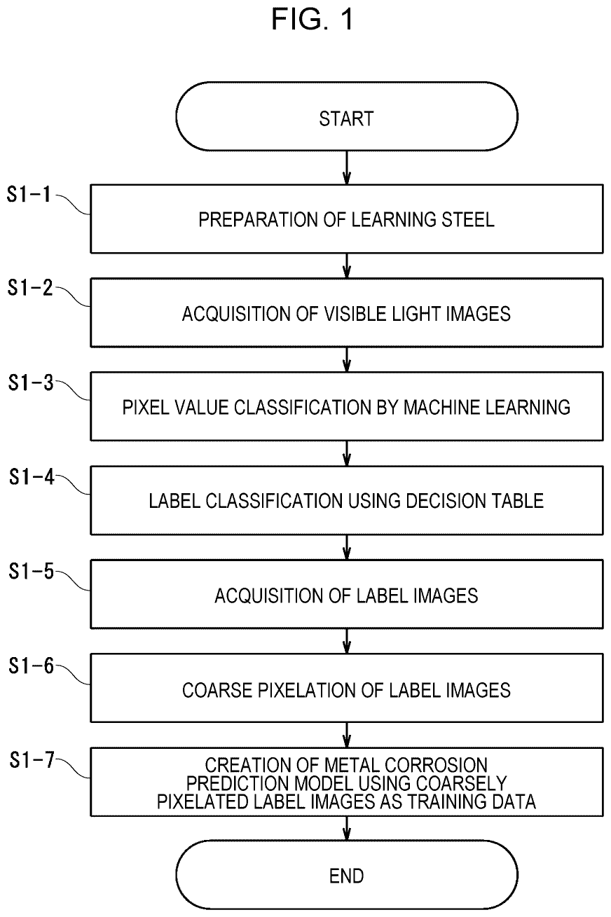

FIG. 1 is a flowchart showing a method for creating a prediction model for corrosion of metals according to an embodiment.

When creating a prediction model for corrosion of metals, a learning steel is prepared first (step S1-1). A corrosion experiment has been conducted on the learning steel in advance to identify the type of a substance(s) to be produced during a corrosion process. When the type of a substance(s) to be produced during a corrosion process is unknown, a corrosion experiment such as a salt spray test may be actually conducted on the steel, and a substance(s) produced during the corrosion process may be identified through an XRD analysis (analysis by X-ray diffractometry) or the like.

Next, visible light images are acquired (step S1-2). A salt spray test was performed on the learning steel, and images indicating the corrosion state of the steel surface were taken 1, 3, 6, 8, 24, 48, and 72 hours after the start of the spray to acquire a plurality of (7) visible light images. The learning steel was a carbon steel having a thickness of 2 mm and a size of 30 mm×30 mm. The substances produced during the corrosion process were identified as α-FeOOH, β-FeOOH, and Fe₃O₄ by an XRD analysis (analysis by X-ray diffractometry).

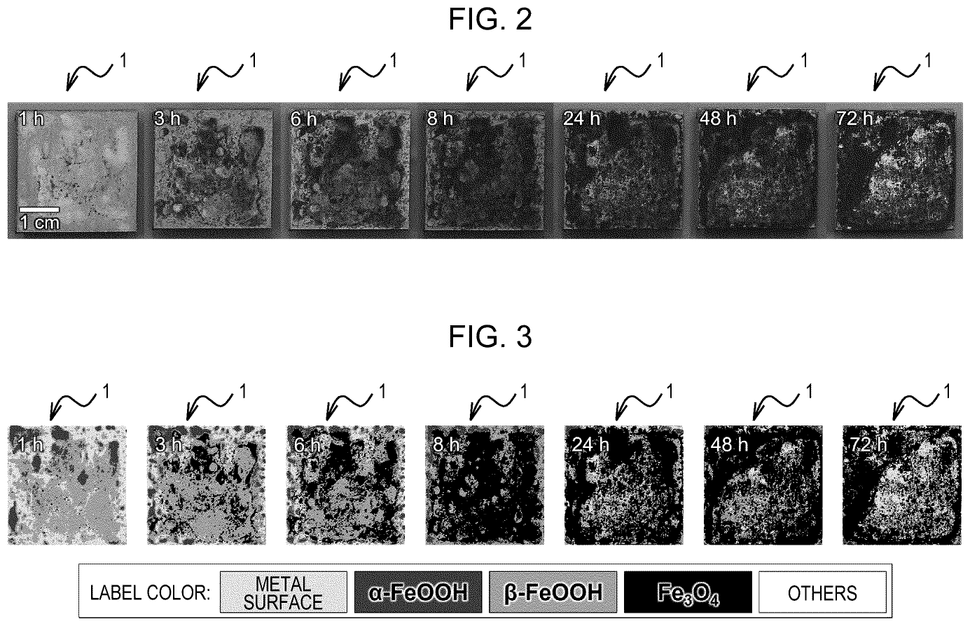

FIG. 2 shows the visible light images of a steel surface 1 1, 3, 6, 8, 24, 48, and 72 hours after the start of corrosion.

The images of FIG. 2 indicate how corrosion of the steel surface 1 progresses over time.

Next, pixel value classification using machine learning is performed (step S1-3). Pixel value classification using machine learning was performed on the acquired visible light images. The pixel value classification was performed by linking the time-series visible light images to create a single linked image as shown in FIG. 2, and subjecting the linked image to unsupervised learning.

In the pixel value classification, three-dimensional RGB pixel values, expressed as coordinate values in color space of 8-bit color (256 gradations×3 colors), were classified into three-level intensity classes for each dimension (R value, G value, B value) using the k-means method. The thus-obtained classes are termed R0, R1, R2, G0, G1, G2, B0, B1, and B2 in order of decreasing intensity for each dimension (R value, G value, B value). For example, a color distribution is used which uses three discrete classes of high intensity (170-255), medium intensity (85-169), and low intensity (0-84) for each of RGB (red, green, blue) dimensions. When the R value, G value, and B value of a color are all high, the color is expressed as R0-G0-B0. In this manner, a color distribution having 3 R classes×3 G classes×3 B classes, for a total of 27 sets, was acquired.

While an example embodiment, which uses three-dimensional RGB values as coordinate values in color space, has been described above, the present invention is not limited to such an embodiment; other coordinate values in color space such as three-dimensional HSV (hue, saturation, value) values may be used. Thus, a color distribution is created by classifying the multi-dimensional coordinate values in color space of visible light images into a predetermined number of classes for each dimension by machine learning.

Next, label classification is performed using a decision table (step S1-4), and label images are acquired (step S1-5). A label distribution is created based on the above-obtained color distribution of the coordinate values in color space of the visible light images. In this embodiment, a decision table was set which reduces the acquired color distribution having 27 sets to five types (five colors) of labels (see Table 1 below). The number of labels (five types in this embodiment) was set according to the number of the types of substances present in the corrosion process of the steel surface. In particular, each label corresponds to one of the following five substances: the steel surface, α-FeOOH, β-FeOOH, Fe₃O₄, and others. The “others” correspond to an image of a substance other than the test specimen and pixels which cannot be properly classified.

TABLE 1

| Corrosion state labels | Sets of RGB-value classes |
|---|---|
| Metal surface | R0-G0-B0, R1-G0-B0, R1-G1-B0 |
| α-FeOOH | R0-G0-B2, R0-G1-B2, R1-G1-B2 |
| β-FeOOH | R0-G0-B1, R0-G1-B1, R0-G1-B2, |
| | R1-G0-B1, R1-G1-B1, R1-G1-B2, |
| | R1-G2-B1, R2-G1-B2, R2-G2-B1 |
| Fe₃O₄ | R2-G2-B2 |
| Others | Other 11 sets |

The decision table was created so that coloration with the 5-color labels corresponded to the original visible light images by referring to the distribution of substances based on the results of XRD analysis. Label classification was performed according to the thus-set decision table, and label images were acquired in which each pixel was assigned a corresponding label.

FIG. 3 shows label images of the steel surface 1 1, 3, 6, 8, 24, 48, and 72 hours after the start of corrosion.

Compared to FIG. 2, it is easier with FIG. 3 to visually perceive the types and the positions of substances appearing on the steel surface 1. Thus, the distribution of the metal surface, α-FeOOH, β-FeOOH, Fe₃O₄, and other substances on the steel surface 1 can be accurately perceived.

Next, the label images are subjected to coarse-pixelation (step S1-6). The label images of FIG. 3 were coarsely pixelated. In particular, each of the above-obtained label images was divided into a plurality of coarse pixel regions. In each coarse pixel region, a label with the highest frequency of appearance among the labels contained in the coarse pixel region was taken as a label of that coarse pixel region.

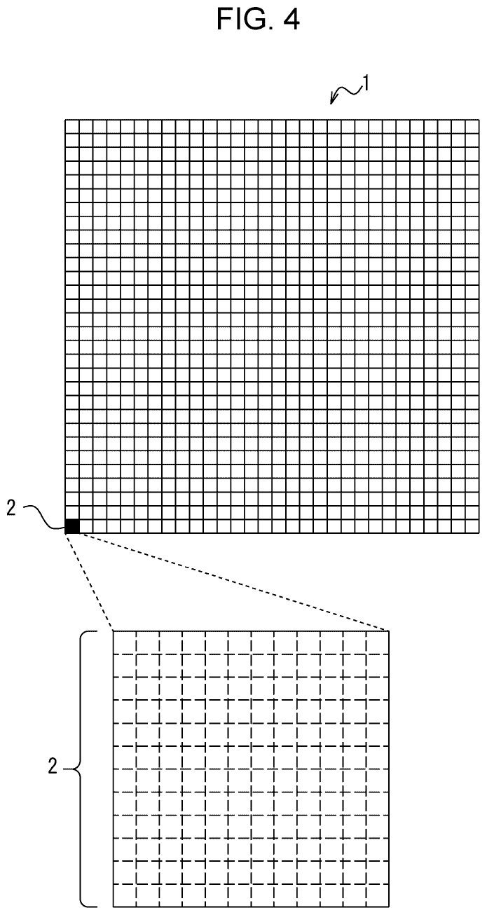

FIG. 4 is a diagram illustrating coarse-pixelation of a label image.

Each of the five-color label images was trimmed into a square of approximately 360 pixels×360 pixels, and then coarsely pixelated to 30 pixels×30 pixels. Thus, the label image was subjected to a process (coarse-pixelation process) to reduce the resolution so that approximately 12 pixels×12 pixels=approximately 144 pixels constituted one coarse pixel region 2. Each area bounded by solid lines in the upper diagram of FIG. 4 corresponds to a coarse pixel region 2, and each area bounded by broken lines in the lower diagram of FIG. 4 corresponds to a pixel before coarse-pixelation. One label was assigned to each coarse pixel region 2. The label was the one whose appearance frequency was the highest among the labels contained in the coarse pixel region 2 (144 pixels). When the appearance frequencies are the same, the order of priority is set as follows: metal surface>β-FeOOH>α-FeOOH>Fe₃O₄.

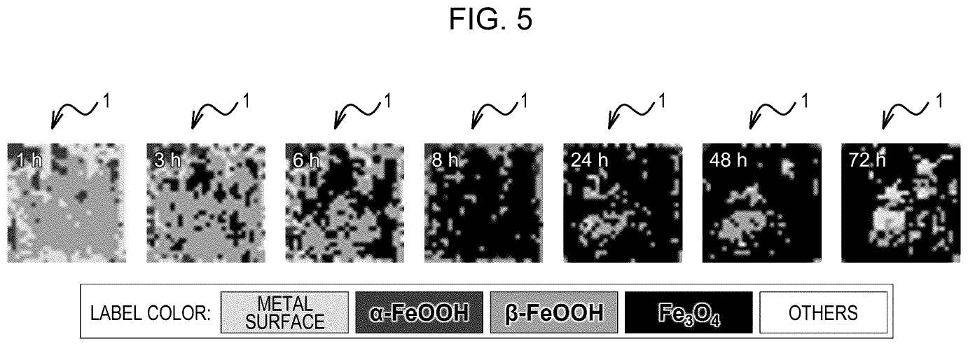

FIG. 5 shows coarsely pixelated label images of the steel surface 1, 3, 6, 8, 24, 48, and 72 hours after the start of corrosion.

Compared to FIG. 3, it is further easier with FIG. 5 to visually perceive the types and the positions of substances appearing on the steel surface 1. Thus, the distribution of the metal surface, α-FeOOH, β-FeOOH, Fe₃O₄, and other substances on the steel surface 1 can be more accurately perceived.

Finally, a prediction model for corrosion of metals is created using the coarsely pixelated label images as training data (step S1-7). A prediction model for corrosion of metals, which predicts a future change in the corrosion state of a steel surface to be predicted from a visible light image of the steel surface, was created using the above-obtained coarsely pixelated label images as training data. The creation of the prediction model for corrosion of metals was performed by subjecting the coarsely pixelated label images to supervised machine learning using the random forest method. A label value of one pixel was predicted from label values of nine pixels. The supervised machine learning may be performed by a known method such as a neural network, SVM (support vector machine), or decision tree, or a combination thereof.

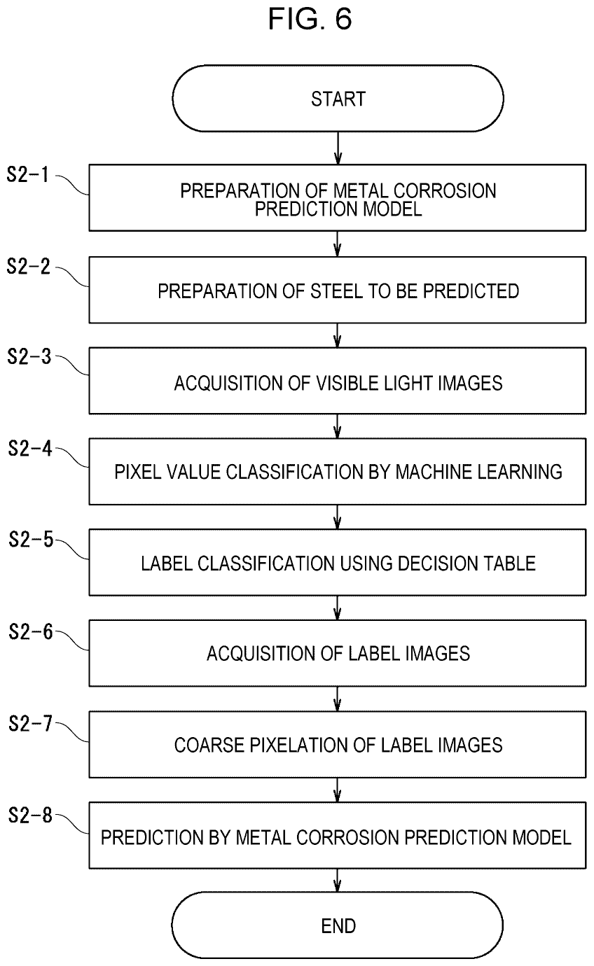

FIG. 6 is a flowchart showing a metal corrosion prediction method using the prediction model for corrosion of metals thus created.

First, in preparation steps, the learned prediction model for corrosion of metals thus created is prepared (step S2-1), a steel to be predicted is prepared (step S2-2), and the steel is imaged to acquire a visible light image (step S2-3). Next, in a color distribution acquisition step, the acquired visible light image is subjected to pixel value classification by machine learning (step S2-4). Next, in a labeling step, label classification is performed using a decision table (step S2-5), and a label image is acquired (step S2-6). Next, in a coarse-pixelation step, the label image is coarsely pixelated (step S2-7). Steps S2-4 to S2-7 are substantially the same as steps S1-3 to S1-6 shown in FIG. 1.

Next, in a corrosion prediction step, the above-obtained coarsely pixelated label image is input to the prediction model for corrosion of metals, and the prediction model outputs a future change in the corrosion state of the steel surface (step S2-8).

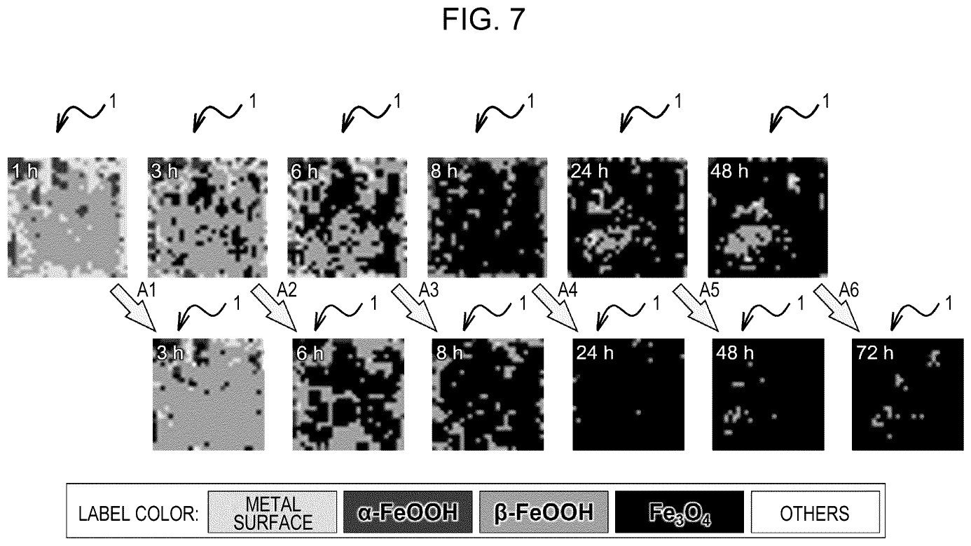

FIG. 7 shows images illustrating examples of the results of prediction by the prediction model for corrosion of metals.

In this embodiment, a corrosion change 3 hours after the start of corrosion was predicted using a coarsely pixelated label image 1 hour after the start of corrosion (see arrow A1). Similarly, a corrosion change 6 hours after the start of corrosion was predicted using a coarsely pixelated label image 3 hours after the start of corrosion (see arrow A2). Similarly, a corrosion change 8 hours after the start of corrosion was predicted using a coarsely pixelated label image 6 hours after the start of corrosion (see arrow A3). Similarly, a corrosion change 24 hours after the start of corrosion was predicted using a coarsely pixelated label image 8 hours after the start of corrosion (see arrow A4). Similarly, a corrosion change 48 hours after the start of corrosion was predicted using a coarsely pixelated label image 24 hours after the start of corrosion (see arrow A5). Similarly, a corrosion change 72 hours after the start of corrosion was predicted using a coarsely pixelated label image 48 hours after the start of corrosion (see arrow A6). It is also possible to predict one or more future images from two or more past images.

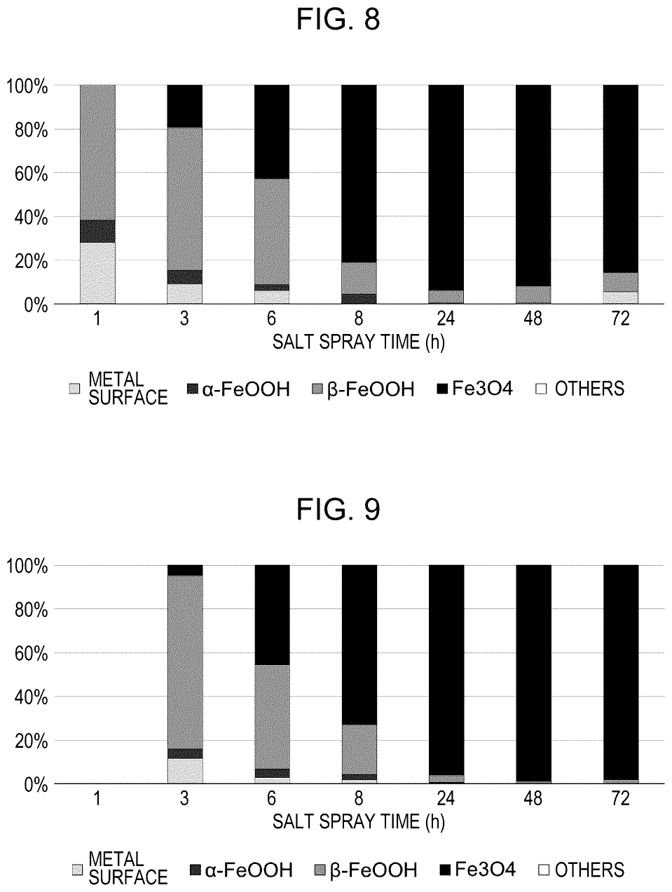

FIG. 8 is a graph showing the occupied area ratio of each label in an experimental steel. FIG. 9 is a graph showing the predicted occupied area ratio of each label.

A plurality of pieces of steel were prepared, some of which were used as a learning steel and the rest were used as a steel to be predicted. These were placed flat in a combined cycle testing machine and subjected to a salt spray test using a 5 wt % NaCl aqueous solution at 35° C., and visible light images of the surface were taken after a lapse of 1, 3, 6, 8, 24, 48, and 72 hours. A future corrosion change was predicted from the visible light images in the above-described manner, and compared with the experimental results. The comparison was performed not for the entire coarse pixel regions 2 (30×30 pixels) but for the central portion (28×28 pixels) thereof.

As a result of the comparison between FIGS. 8 and 9, it was found that the prediction of a change in the area ratios of the five-color labels was approximately accurate, and the accuracy rate of each pixel, represented by one of the five-color labels, was 73%. The comparative data thus indicates the effectiveness of the method for predicting metal corrosion of this embodiment.

The method of this embodiment has the following advantageous effects.

A label image that indicates a distribution of labels in place of a distribution of substances, which is seen as a color distribution in a visible light image of a steel surface 1, is used as training data. In particular, the pixel value of each pixel in the visible light image of the steel surface 1 is replaced with a label set according to a substance produced on the steel surface 1 during a corrosion process. Thus, the method predicts not the corrosion state of an unspecified number of substances, but the corrosion state of a small number of specified substances. This enables a highly accurate prediction. Further, a prediction model for corrosion of metals can be created by preparing information on the types of substances which are to be precipitated during a corrosion process on the steel surface 1 and which have been identified by any method in advance, and time-series visible light images which indicate the corrosion process on the metal surface. Thus, the present method can be performed in a very simple manner. In particular, the method directly learns and predicts a time-dependent change in the label distribution, and therefore does not require information on the material of the steel surface 1, the corrosion environment, etc.

The present method uses a color distribution obtained by classifying coordinate values in color space, which can be a huge amount of data, into a predetermined number of classes. This can facilitate handling of the data.

The use of unsupervised learning for the pixel value classification enables classification which is less susceptible to variations in brightness and color tone among images and which is highly tolerant of changes in the imaging environment. In other words, since visible light images may vary in brightness and color tone depending on the imaging environment, the use of supervised learning for the pixel value classification may result in incorrect classification due to a change in the imaging environment.

Further, the use of a coarsely pixelated label image having coarse pixel regions 2 can reduce the complexity of the label distribution, making it possible to perform simpler estimation. The coarse-pixelation steps (step S1-6 and S2-7) may be omitted as necessary.

While the present invention has been described with reference to embodiments and their variations, the present invention is not limited to such embodiments. Changes and modifications may be made to the embodiments without departing from the spirit and scope of the present invention. For example, an appropriate combination of features of some of the embodiments may constitute an embodiment of the present invention.

What is claimed is:

1. A method for creating a prediction model for corrosion of metals, comprising using as training data a plurality of label images which indicate a distribution of substances, in terms of a distribution of labels, in a plurality of time-series visible light images indicating a corrosion process on a metal surface to create a prediction model for corrosion of metals which predicts a future change in a corrosion state of a metal surface to be predicted from a visible light image of the metal surface,
wherein the number of the labels is set according to the number of types of the substances present in the corrosion process of the metal surface, and wherein the distribution of the labels is created based on a color distribution of coordinate values in color space of the visible light images.

2. The method for creating a prediction model for corrosion of metals according to claim 1, wherein the color distribution is created by classifying multi-dimensional coordinate values in color space of the visible light images into a predetermined number of classes for each dimension by machine learning.

3. The method for creating a prediction model for corrosion of metals according to claim 2, wherein the machine learning is unsupervised learning.

4. A method for predicting metal corrosion, comprising:
a preparation step of preparing a learned prediction model for corrosion of metals using as training data a plurality of label images which indicate a distribution of substances, in terms of a distribution of labels, in a plurality of time-series visible light images indicating a corrosion process on a metal surface, wherein the number of the labels is set according to the number of types of the substances present in the corrosion process of the metal surface, and wherein the distribution of the labels is created based on a color distribution of coordinate values in color space of the visible light images;
a color distribution acquisition step of acquiring a color distribution based on coordinate values in color space from a visible light image indicating a corrosion process on a metal surface to be predicted;
a labeling step of acquiring a label image which indicates a distribution of labels created based on the color distribution; and
a corrosion prediction step of inputting the label image to the prediction model for corrosion of metals to predict a future change in a corrosion state of the metal surface.

5. The method for predicting metal corrosion according to claim 4, wherein the color distribution is created by classifying multi-dimensional coordinate values in color space of the visible light image into a predetermined number of classes for each dimension by machine learning.

6. The method for predicting metal corrosion according to claim 5, wherein the machine learning is unsupervised learning.

7. The method for predicting metal corrosion according to claim 4, further comprising a coarse-pixelation step of dividing the label image acquired in the labeling step into a plurality of coarse pixel regions, and taking a label with a highest frequency of appearance among labels contained in each of the coarse pixel regions as a label of that coarse pixel region.

8. The method for predicting metal corrosion according to claim 5, further comprising a coarse-pixelation step of dividing the label image acquired in the labeling step into a plurality of coarse pixel regions, and taking a label with a highest frequency of appearance among labels contained in each of the coarse pixel regions as a label of that coarse pixel region.

9. The method for predicting metal corrosion according to claim 6, further comprising a coarse-pixelation step of dividing the label image acquired in the labeling step into a plurality of coarse pixel regions, and taking a label with a highest frequency of appearance among labels contained in each of the coarse pixel regions as a label of that coarse pixel region.
